(19) United States
(12) Patent Application Publication
(10) Pub. No.: US 2020/0165643 A1
(43) Pub. Date: May 28, 2020

(54) PROCESS FOR REDUCING BIOCHEMICAL OXYGEN DEMAND OF FOOD AND BEVERAGE PROCESSING EFFLUENT

(71) Applicants: Derrick Risner, Davis, CA (US); Sara A. Pace, Davis, CA (US)

(72) Inventors: Derrick Risner, Davis, CA (US); Sara A. Pace, Davis, CA (US)

(21) Appl. No.: 16/694,591

(22) Filed: Nov. 25, 2019

Related U.S. Application Data
(60) Provisional application No. 62/771,165, filed on Nov. 26, 2018.

(51) Int. Cl. C12P 17/02; C12P 7/40; C12F 3/02; B01D 11/04; C02F 11/04

(52) U.S. Cl. CPC C12P 17/02; C12P 7/40; C02F 2103/327; B01D 11/0403; C02F 11/04; C12F 3/02

(57) ABSTRACT

A process and system for reducing biochemical oxygen demand (BOD) of food and beverage processing effluents.

This application claims the benefits of U.S. Provisional Patent Application No. 62/771,165 filed on Nov. 26, 2018, the contents of which are incorporated herein by reference.

### FIELD OF THE INVENTION

The present invention relates to a process for reducing the biochemical oxygen demand (BOD) of food and beverage processing effluent and more specifically reducing the BOD of whey and/or whey permeate.

### BACKGROUND OF THE INVENTION

Food and beverage processing facilities often generate an effluent stream that is high in sugar content. This effluent must be processed to reduce the sugar content before disposal because the oxygen demand created by the breakdown of the high sugar content in the effluent will causes drastic oxygen depletion in the disposal media resulting in detrimental environmental impact.

For example, a commonly known food processing effluent is whey. Whey is a by-product from cheese manufacture and typically comprises about 94% water, about 0.05% fat, about 0.6% protein and about 4.5% lactose. Whey also contains free amino nitrogen, minerals and vitamins in concentrations capable of supporting microbial growth. There are two main types of whey. The first known as “sweet whey” is typically produced during the production of lower acidic cheeses such as cheddar and mozzarella. The second type of whey is known as “acid whey” and is typically produced during the production of high acid cheeses and dairy products such as cottage cheese, cream cheese and Greek yogurt. Acid whey may have twice the calcium content and more than 10 times the lactic acid content of sweet whey. Approximately 9 kilograms of whey is produced per kilogram of cheese manufactured and yearly whey production rates reach up to 180 million tons. Whey has a high BOD concentration that has been reported to range from 30,000 to 50,000 mg/l BOD when compared to other waste stream such as raw sewage domestic sewage which has a BOD concentration of about 300-500 mg/l. Whey's high BOD concentration is primarily due to its lactose content. Due to whey's BOD concentration and environmental regulations, whey must be processed or disposed of in manner which reduces its BOD content. Current commercial whey processing technology employs numerous separation techniques such as centrifugation to remove the fat, membrane filtration to remove the protein and multi-step evaporation/crystallization processes to remove the lactose. This processing technology has high capital costs and is often not capable of handling the mineral and acid content present in acid whey.

Part of the cost of processing food and beverage processing effluent such as whey, maybe recoverable if the processing derivatives have commercial value. For example, there is a demand and commercial uses for whey protein so the cost to remove the whey protein from whey can often be justified, however, the whey permeate (whey with the protein and fat removed) still has a composition of about 95% water and about 4.5% lactose. The whey permeate also retains the free amino nitrogen, vitamins and minerals necessary for microbial growth after protein recovery. Although, the lactose can be removed from the whey and/or whey permeate by multistep evaporation and crystallization processes, the required equipment can cost millions of dollars and sale of the recovered lactose often does not justify the equipment expense.

The BOD concentration of food and beverage processing effluent that are high is sugar content such as the lactose in whey and whey permeate have been reduced by a fermentation process wherein the sugar is converted to ethanol and CO₂. The ethanol has been used for fuel and purified for use in alcoholic spirits such as gin and vodka or use in drug and medical processes. This fermentation process is described in publications such as Risner D, et al., “Volatile Aroma Composition of Distillates Produced From Fermented Sweet and Acid Whey,” J Dairy Sci (2019); 102(1):202-10; Hughes P, et al., “Whey to Vodka,” In: Whey—Biological Properties and Alternatives. 1st ed. London: IntechOpen; 2018 (available from: http://dx.doi.org/10.5772/intechopen.81679; and Hamilton R, et al., “The Manufacture of Ethanol from Whey” July 2018 (available from: https://nzic.org.nz/app/uploads/2017/10/3H.pdf). The foregoing references are incorporated herein by reference.

The whey fermentation treatment process still has drawbacks because the it requires additional holding/fermentation vessels, centrifugation equipment and distillation equipment. Additionally the fermentation process produces a yeast/microbial biomass that has little to no value and must also be disposed.

There is a need to improve the processes for reducing the BOD concentration of food and beverage processing effluent.

### SUMMARY OF THE INVENTION

The foregoing need is met by the present invention which are processes and systems for reducing the BOD concentration of food and beverage processing effluent and particularly, reducing the BOD concentration of whey and/or whey permeate.

One embodiment of the present invention is a process and system for reducing the BOD concentration of food and beverage processing effluent and particularly, reducing the BOD concentration of whey and/or whey permeate comprising:
(i) contacting the process effluent with one or more microorganisms that have been genetically engineered to produce secondary metabolites wherein the secondary metabolites are selected from the group consisting of terpenoids, cannabinoids, cannabinoid producing enzymes, alkaloids or combinations thereof;
(ii) allowing the process effluent and microorganisms to react or ferment creating a reaction mass comprising the process effluent with a reduced BOD, the microorganisms and the secondary metabolites;
(iii) removing the secondary metabolites from the reaction mass.

The reaction or fermenting of the process effluent and microorganisms may also generate additional reactants such as ethanol and CO₂. These additional reactants if present may also be removed from the reaction mass. The removed reactants may be isolated, purified and/or further processed.

Another embodiment of the present invention is a process and system for reducing the BOD concentration of food and beverage processing effluent and particularly, reducing the BOD concentration of whey and/or whey permeate comprising:
(i) contacting the process effluent with one or more microorganisms that have been genetically engineered to produce secondary metabolites wherein the secondary metabolites are selected from the group consisting of terpenoids, cannabinoids, cannabinoid producing enzymes, alkaloids or combinations thereof;
(ii) allowing the process effluent and microorganisms to react or ferment creating CO₂ and a reaction mass comprising the process effluent with a reduced BOD, the microorganisms and the secondary metabolites
(iii) capturing the CO₂ generated in step (ii); and
(iv) using the captured CO₂ in a supercritical CO₂ extraction process to extract the secondary metabolites from the reaction mass of step (ii).

A further embodiment of the present invention is a process and system for reducing the BOD concentration of food and beverage processing effluent and particularly, reducing the BOD concentration of whey and/or whey permeate comprising:
(i) contacting the process effluent with hexanoic acid and one or more microorganisms that have been genetically engineered to produce a cannabinoid;
(ii) allowing the process effluent, hexanoic acid and microorganisms to react or ferment creating a reaction mass comprising the process effluent with a reduced BOD, the microorganisms and a cannabinoid;
(iii) removing the cannabinoid from the reaction mass of step (ii).

If CO₂ is produced during the reaction or fermentation it may be removed from the reaction vessel/reaction mass and used in a supercritical CO₂ extraction process to extract the cannabinoids from the reaction mass of step (ii). In certain aspects of this embodiment the hexanoic acid employed may be prepared by: (a) contacting/fermenting a first process effluent with a high BOD concentration with genetically engineered microorganisms that produces hexanoic acid to produce hexanoic acid and a hexanoic acid reaction mass; (b) removing the hexanoic acid produced in step (a) for use in step (i). Alternatively, the hexanoic acid reaction mass of step (a) may be added to the reaction mass of step (i) without the removal, isolation or concentrating of the hexanoic acid.

The removed secondary metabolites such as the cannabinoids described in the above processes may be isolated, purified and/or further processed for sale.

The reaction mass comprising the processing effluent with the reduced BOD and the microorganisms as well as any required nutrients, minerals etc. prepared in any of the above embodiments may be further processed by separating the process effluent with a reduced BOD concentration from the biomass and using the biomass to generate energy that is used in the above processes. For example, the biomass may be subjected to anaerobic digestion to produce a biogas such as CH₄ that can be used to power combined heat and power systems and electric generators which in turn power the pumps that move the various reactants and/or reaction masses, or equipment that generates the supercritical CO₂. The biogas may also be used to create steam or hot water to heat the various reaction vessels.

### BRIEF DESCRIPTION OF THE DRAWING

FIG. 1 is a schematic diagram showing one embodiment of the present invention wherein the secondary metabolites are removed from the reaction mass with a supercritical CO₂ extraction.

FIG. 2 is a schematic diagram showing one embodiment of the present invention wherein hexanoic acid is generated by contacting/fermenting a first process effluent with genetically engineered microorganisms that produce hexanoic acid, separating the hexanoic acid from the reaction mass and adding the separated hexanoic acid to a reacting vessel containing a food or beverage process effluent with a high BOD concentration and a genetically engineered microorganism that produces a cannabinoid.

FIG. 3 is a schematic diagram showing one embodiment of the present invention wherein hexanoic acid is generated by contacting/fermenting a first process effluent with genetically engineered microorganisms that produce hexanoic acid and adding a reaction mass with the hexanoic acid and process effluent (with or without a biomass) to a reacting vessel containing a food or beverage process effluent with a high BOD concentration and a genetically engineered microorganism that produces a cannabinoid.

FIG. 4 is a schematic diagram showing one embodiment of the present invention wherein the reaction mass comprising the processing effluent with a reduced BOD concentration and the microorganisms as well as any required nutrients, minerals etc. is separated and the biomass is employed subjected to anaerobic digestion to create CH₄.

### DETAILED DESCRIPTION OF THE INVENTION

Before the present invention is further described, it is to be understood that this invention is not limited to the particular embodiments described. It is also to be understood that the terminology used herein is for the purpose of describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein the term “food and beverage processing effluent” and “processing effluent” and “effluent” may be used interchangeably unless specifically indicated to the contrary. These terms encompass a liquid by-product or co-product generated during the manufacturing or processing of a food or beverage. In certain aspects of the invention, the food and beverage processing effluent with a high BOD concentration will exhibit a BOD of greater than 750 mg/L, greater than 1,000 mg/L greater than 2,500 mg/L, greater than 5,000 nm/L, greater than 7,500 mg/L, greater than 10,000 mg/L; greater than 12,500 mg/L, greater than 15,000 mg/L, greater than 17,500 mg/L, greater than 20,000 mg/L and greater than 25,000 mg/L. The processes and systems of the present invention should reduce the BOD of the processing effluent by at least 5%, 10%, 15%, 20%, 25%, 30%, 35%, 40%, 45%, 50%, 55%, 60%, 65%, 70%, 75%, 80%, 85%, 90% or 95% or more. In certain embodiments the processed effluent discharged from the process and systems of the present invention should have a BOD concentration of less than 500 mg/L, less than 400 mg/L, less than 300 mg/L, less than 200 mg/L or less than 100 mg/L.

As used herein the term “whey” refers a liquid by-product or co-product generate during the production of cheese and dairy products. Unless specifically stated, “whey” includes all types and classifications of why including sweet whey and acid whey. The term “whey permeate” includes whey that has been filtered or processed to remove specific components, typically proteins and fat.

As used herein the term “secondary metabolite” refers to one or more of the products produced when a microorganism such as a bacteria, cyanobacteris, filamentous fungi, algae or yeast contacts a carbon source, particularly a sugar such as dextrose, fructose, glactose, glucose, lactose, maltose, sucrose, xylose etc. and the microorganism metabolizes the carbon source, particularly a sugar, into new compounds such as terpenoids, cannabinoids, cannabinoid producing enzymes and alkaloids. This process is sometime referred to as “fermentative production”. Secondary metabolites do not include the well-known fermentative production products such as CO₂ and ethanol although these well-known fermentative products may also be produced during the production of the desired or target secondary metabolites of the present invention.

The term “reaction mass” is used to described the contents of a reaction vessel and may include process effluent, microorganisms for reacting with or fermenting the process effluent, and any other materials such a pH adjusting agents, or incubation materials need to assist in the growth and propagation of the microorganisms. The reaction mass may also include the well-known metabolites of the fermentation process and the secondary metabolites. The term “biomass” as used herein refers to material produced by microorganisms as well as the microorganisms, essentially the solids of the fermentation process. Biomass can contain cells, microbes, viruses and/or intracellular contents as well as extracellular material including, for example, compounds that are secreted by a cell, such as secreted secondary metabolites. The biomass may be part of the reaction mass.

The genetically engineered microorganisms that produce the desired or target secondary metabolites of the present invention are known in the art or can be prepared a person of ordinary skill in the art of microbial engineering using known techniques. Some examples of the genetically engineered microorganisms that can be used in the present invention are described in U.S. Pat. No. 9,822,384 which describes genetically engineered microorganisms that can produce cannabinoids including genetically modified S. cerevisiae and K. marxianus; U.S. Patent Application Publication No. 2017/0233778 which describes genetically engineered microorganisms that can produce cannabinoid producing enzymes including genetically modified Pichia Pastoris; U.S. Patent Application Publication Nos. 2018/0080035 and 2018/014697 which describe genetically engineered microorganisms that can produce terpenoids; U.S. Patent Application Publication No. 2018/0334692 which describes genetically engineered microorganisms that can produce cannabinoid producing enzymes and cannabinoids; International Patent Application Publication Nos. WO 2017/139496 and WO 2019/014490 which describe genetically engineered microorganisms that can produce cannabinoid producing enzymes, cannabinoid precursor such as hexanoic acid and olivetolic acid and cannabinoids; Luo X, et. al., “Complete Biosynthesis of Cannabinoids and Their Unnatural Analogues in Yeast,” Nature, March 2019; 567(7746):123-6 (http://www.nature.com/articles/s41586-019-0978-9) which describes genetically engineered microorganisms that can produce cannabinoid producing enzymes, cannabinoid precursor and cannabinoids; Cheon Y, e.t al., “A Biosynthetic Pathway for Hexanoic Acid Production in Kluyveromyces Marxianus,” J Biotechnol, July 2014 63(3): 223-24 (http://www.ncbi.nlm.nih.gov/pubmed/24768798) which describe genetically engineered microorganisms that can produce hexanoic acid; Yang J, et. al., “Metabolic Engineering of Escherichia Coli for the Biosynthesis of Alpha-Pinene,” Biotechnol Biofuels, April 2013; 6(1):60 (http://biotechnologyforbiofuels.biomedcentral.com/articles/10.1186/1754-6834-6-60) which describe genetically engineered microorganisms that can produce α-pinene; Ehrenworth A M, et. al., “Accelerating the Semisynthesis of Alkaloid-Based Drugs Through Metabolic Engineering,” Nat Chem Biol., March 2017; 13 (3): 249-58 (http://www.nature.com/articles/nchembio 0.2308) which describes genetically engineerd microoganisms that can produce the N-demethylnarwedine which can be converted to the alkaloid galantamine; Zhuang et al., “Monoterpene Production by the Carotenogenic Yeast Rhodosporidium Toruloides,” Microbial Cell Factories 2019; 18(1):1-(https://microbialcellfactories.biomedcentral.com/track/pdf/10.1186/s12934-019-1099-8) which describes microorganisms which produce terpenoids; Kang et. al., “Biosynthesis of Pinene From Glucose Using Metabolically-Engineered Corynebacterium Glutamicum,” Biotechnol. Lett. 2014, 36:2069-2077 which describes the production of terpenoids; Xie et, al., “Mining Terpenoids Production and Biosynthetic Pathway in Thraustochytrids” Bioresource Technology 2017; 244: 1269-1280; which describes the microalgae/protists production of terpenes. The contents of the foregoing patents, patent applications and publications are incorporated herein by reference.

The reaction or fermentation of the processing effluent with a high BOD concentration and genetically engineered microorganisms may occur in an appropriate reaction vessel or bioreactor. In some embodiments, the genetically engineered microorganism can be added to the reaction vessel or bioreactor as spores and/or any kind of dormant cell type of any isolated microorganism described herein, for example, in a dry state. In some embodiments, the addition of the process effluent to the reaction vessel can lead to activation of the dormant cells, for example, to the germination of a yeast spore, and subsequent conversion of the sugar source in the process effluent to one or more of the desired or targeted secondary metabolites such as a cannabinoid, cannabinoid precursor or a cannabinoid producing enzyme. In some embodiments, the genetically engineered microorganisms are activated prior to the addition of the processing effluent with the high BOD concentration. The prior activation of the genetically engineered microorganisms may occur in the reaction vessel wherein the reaction or fermentation process occurs or alternatively the prior activation may in a separate reaction vessel prior to the addition to the reaction vessel wherein the reaction or fermentation process occurs.

In some embodiments, the genetically engineered microorganisms described are contacted with the aqueous process effluent such as whey or whey permeate in a bioreactor, and the generated/secreted fermentation products including the secondary metabolites, form an organic phase that can be separated from the aqueous phase. The term “organic phase” can refer to a liquid phase comprising a non-polar, organic compound, including, for example, a cannabinoid, a cannabinoid precursor, and/or a non-polar lipid. An organic phase described herein can further contain microorganisms or other compounds also found in aqueous phase.

Methods useful for removing or separating the organic phase and/or secondary metabolites from the reaction mass are well known to those of ordinary skill in the art. In some embodiments, the organic phase is continuously or semi-continuously siphoned off. In some embodiments, a reaction vessel or bioreactor can comprise a separator that can be used, to continuously or semi-continuously extract the organic phase from the inorganic phase. In certain embodiments the separation employs supercritical CO₂ extraction, preferably a counter current supercritical CO₂ extraction step wherein at least a portion of the CO₂ used in the supercritical CO₂ extraction has been obtained from the CO₂ generated by the reaction of the genetically engineered microorganisms and the process effluent with the high BOD concentration or generated by an earlier reaction/batch of the genetically engineered microorganisms and the process effluent with the high BOD concentration.

In some embodiments, the desired or target secondary metabolites of the present invention can accumulate in a cell according to aspects described herein. In some embodiments, a cell that accumulates a desirable amount of the desired or target secondary metabolites can be separated continuously or semi-continuously from a bioreactor. Non-limiting chemical separation methods include centrifugation, sedimentation, and filtration. Cell separation can further be affected based on a change in physical cell characteristic, such as cell size and cell density, by methods well known to those skilled in the art. The accumulated desired or target secondary metabolites can subsequently be extracted from the respective cells using standard methods of extraction well known to those skilled in the art. Non-limiting extraction methods include liquid-liquid solvent extraction. Additional non-limiting examples of cell extraction methods include the application of enzymes, detergents, heat, pressure, and mechanical action some of which are described in WO 2017/139496 which are incorporated herein by reference. In some embodiments, the extracted desired or target secondary metabolite can be further refined using additional purification methods well known in the art such as fractional distillation, crystallization, solvent extractions, salt formation or chromatographic techniques.

Typically the genetically engineered microorganisms used in the present invention are grown at a temperature in the range of about 25° C. to about 70° C., preferably about 25° C. to about 40° C. in an appropriate medium. Suitable growth media that may be used in the present invention are common commercially prepared media such as Luria Bertani (LB) broth, M9 minimal media, Sabouraud Dextrose (SD) broth, Yeast medium (YM) broth, (Ymin) yeast synthetic minimal media, and minimal media as described herein, such as M9 minimal media. Other defined or synthetic growth media may also be used, and the appropriate medium for growth of the particular microorganism will be known by one skilled in the art of microbiology or bio-production science. In various embodiments a minimal media may be developed and used that does not comprise, or that has a low level of addition of various components, for example less than 10, 5, 2 or 1 g/L of a complex nitrogen source including but not limited to yeast extract, peptone, tryptone, soy flour, corn steep liquor, or casein. These minimal medias may also have limited supplementation of vitamin mixtures including biotin, vitamin B12 and derivatives of vitamin B12, thiamin, pantothenate and other vitamins. Minimal medias may also have limited simple inorganic nutrient sources containing less than 28, 17, or 2.5 mM phosphate, less than 25 or 4 mM sulfate, and less than 130 or 50 mM total nitrogen. The foregoing temperatures and additional growth media nutrients may be employed during the reaction or fermentation of the high BOD concentration processing effluent with the genetically modified microorganisms, or in the activation and/or propagation of the genetically engineered microorganisms prior to contact with the process effluent with a high BOD concentration.

Suitable pH ranges for the reaction or fermentation of the high BOD concentration processing effluent with the genetically modified microorganisms are between pH 3.0 to pH 10.0, preferably between a pH 5.0 to pH 8.0 is a typical pH range for the initial condition. However, the actual culture conditions for a particular embodiment are not meant to be limited by these pH ranges.

The reaction or fermentation of the high BOD concentration processing effluent with the genetically modified microorganisms may be performed under aerobic, microaerobic, or anaerobic conditions, with or without agitation.

The various embodiments of the present invention may employ a batch type reaction vessel or bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the process effluent with high BOD concentration is inoculated with the desired genetically engineered microorganisms, and bio-production is permitted to occur without adding anything to the system. In batch systems the metabolite and biomass compositions of the system change constantly up to the time the bio-production event is stopped, typically when the desired BOD concentration is obtained. Within batch cultures cells moderate through a static lag phase to a high growth log phase and finally to a stationary phase where growth rate is diminished or halted. If untreated, cells in the stationary phase will eventually die as the sugars in the process effluent are depleted.

A variation on the standard batch system may also be used in embodiments of the present invention wherein additional process effluent with a high BOD concentration is added to the reaction at predetermined times or when predetermined levels of BOD or sugars are obtained in the reaction vessel. Batch and fed-batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. Ollis, McGraw Hill, New York, 1986, herein incorporated by reference for general instruction on bio-production.

Although embodiments of the present invention may be performed in batch mode, or in fed-batch mode, it is contemplated that the invention would be adaptable to continuous bio-production methods. Continuous bio-production is considered an “open” system where a defined amount of the process effluent with the high BOD concentration is added continuously to a bioreactor and an equal amount of conditioned media is removed simultaneously for processing. Continuous bio-production generally maintains the cultures within a controlled density range where cells are primarily in log phase growth. Continuous bio-production is particularly advantageous for because it has less down time associated with draining, cleaning and preparing the equipment for the next bio-production event. Furthermore, it is typically more economical to continuously operate downstream unit operations, such as distillation, than to run them in batch mode.

The various embodiments of the present invention comprise a system for reducing the BOD concentration of process effluent and particularly whey and whey permeates comprising: a fermentation tank suitable for microorganism cell culture; a line for discharging contents from the fermentation tank to an extraction and/or separation vessel; and an extraction and/or separation vessel suitable for removal of the desired or target secondary metabolites from cell culture waste. In various embodiments, the system may include one or more pre-fermentation tanks, distillation columns, centrifuge vessels, back extraction columns, mixing vessels, or combinations thereof.

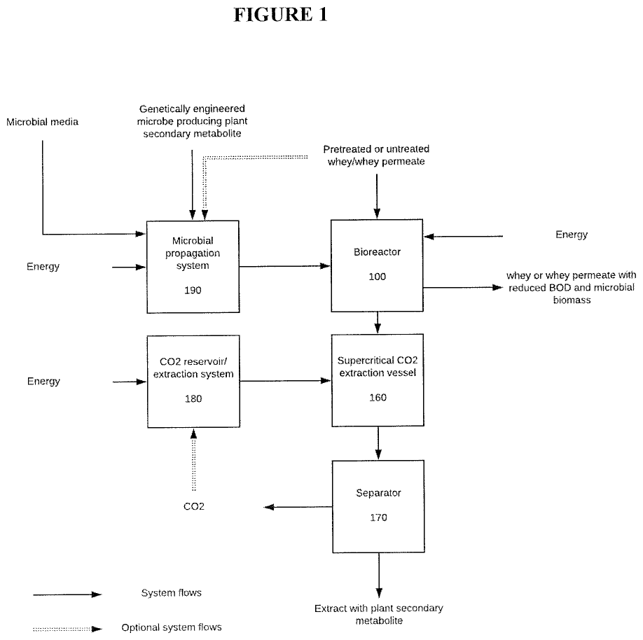

The present invention will be further described by reference to FIGS. 1-4 which are schematic diagrams of various embodiments and aspects of the present invention. This detailed description of the various embodiments is not intended to limit the scope of the present invention. More importantly, a skilled artisan after reviewing this description will understand that various aspects of one or more of the Figures may be combined or eliminated without departing from the scope of the present invention.

FIG. 1 shows the basic aspect of the present invention wherein a process effluent with a high BOD concentration, such as whey or whey permeate is fed into a reaction vessel, bioreactor 100 along with activated genetically engineered microorganisms that have been engineered to produce the desired or target secondary metabolites selected from the group consisting of terpenoids, cannabinoids, cannabinoid producing enzymes, alkaloids or combinations thereof. FIG. 1 further shows one aspect wherein the genetically engineered microorganisms are feed into a microbial propagation system or bioreactor 190 along with a growth media to facilitate the activation and/or growth of the genetically engineered microorganisms. FIG. 1 also depicts alternative embodiments wherein all or a portion of the process effluent with the high BOD concentration is fed into the propagation vessel 190. If all the high BOD process effluent is added to propagation vessel 190, and depending on the size of the propagation vessel, the bioreactor 100 may not be needed. As stated previously, the genetically engineered microorganisms may also be activated prior to the contact with the high BOD process effluent. In this alternative embodiment propagation vessel 190 wherein the high BOD process effluent is added may not be needed. The process effluent with a high BOD concentration and genetically engineered microorganisms are allowed to react until the desired BOD concentration is obtained and the secondary metabolites are removed from the reaction mass. As shown in FIG. 1 the secondary metabolites are removed using a supercritical CO₂ reaction 160. The removed secondary metabolites are further isolated and/or purified using known separation/isolation/purification techniques which are identified in item 170. FIG. 1 further shows that the CO₂ from the supercritical CO₂ extraction may be collected and stored in a CO₂ reservoir for reuse.

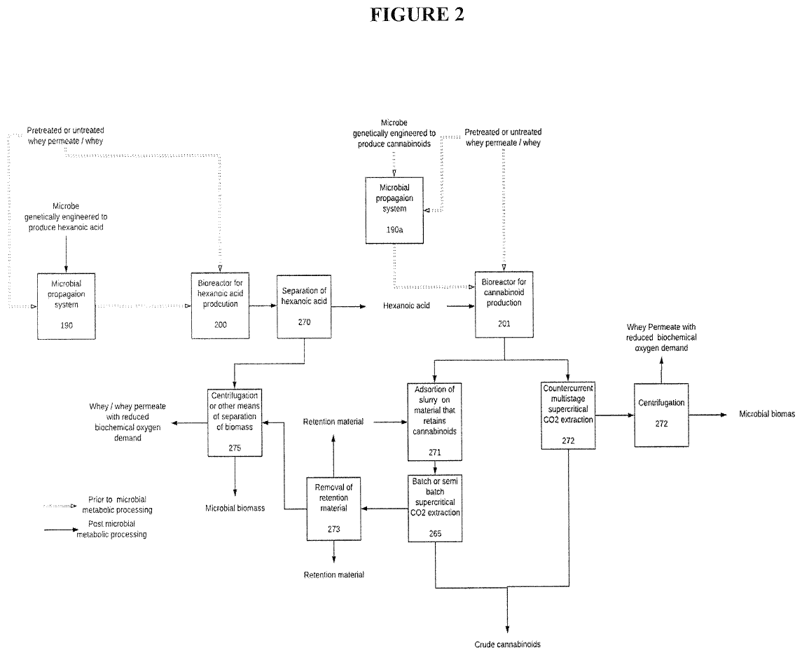

FIG. 2 shows the one embodiment of the present invention wherein a first process effluent with a high BOD concentration is treated with genetically engineered microorganisms to create hexanoic acid and the hexanoic acid is separated from the reaction mass and added to a second process effluent with a high BOD concentration More specifically as shown in FIG. 2, a first process effluent with a high BOD concentration, such as whey or whey permeate, is fed into a reaction vessel, bioreactor 200 along with activated genetically engineered microorganisms that have been engineered to produce hexanoic acid. An example of a suitable genetically engineered microorganism that produces hexanoic acid is described in Cheon Y, et al., “A Biosynthetic Pathway for Hexanoic Acid Production in Kluyveromyces Marxianus,” J Biotechnol, July 2014 63(3): 223-24 (http://www.ncbi.nlm.nih.gov/pubmed/24768798) which is incorporated herein by reference. FIG. 2 further shows one aspect wherein the genetically engineered microorganisms that produce hexanoic acid are feed into a microbial propagation system or bioreactor 190. A growth media to facilitate the activation and/or growth of the genetically engineered microorganisms may be added as shown in FIG. 1 Alternatively, as shown in FIG. 2 all or a portion of the process effluent with the high BOD concentration is fed into the propagation vessel 190. If all the high BOD process effluent is added to propagation vessel 190, and depending upon the size of the propagation vessel, the bioreactor 200 may not be needed. As stated previously, the genetically engineered microorganisms may also be activated prior to the contact with the high BOD process effluent. In this alternative embodiment propagation vessel 190 wherein the high BOD process effluent is added may not be needed. The process effluent with a high BOD concentration and genetically engineered microorganism that produces hexanoic acid are allowed to react until the desired BOD concentration is obtained. The hexanoic acid is separated from the reaction mass in separator 270. The separation of the hexanoic acid may occur by any means commonly used in the industry. In addition, separation of the hexanoic acid may occur prior to, concurrently with or subsequent to the separation of the process effluent with the reduced BOD concentration and the microbial biomass from the reaction mass. The separated hexanoic acid is then added to a reaction vessel or bioreactor 201. As shown in FIG. 2, after the hexanoic acid has been removed, the remaining reaction mass is separated into a process effluent with reduced BOD concentration and microbial biomass by any means known in the industry, preferably by centrifugation 275.

As shown in FIG. 2, a second process effluent with a high BOD concentration, such as whey or whey permeate, is fed into a reaction vessel, bioreactor 201 along with activated genetically engineered microorganisms that have been engineered to produce a cannabinoid. An example of a suitable genetically engineered microorganism that produces a cannabinoid is described in U.S. Pat. No. 9,822,384 which is incorporated herein by reference. FIG. 2 further shows one aspect of this embodiment wherein the genetically engineered microorganisms that produce a cannabinoid are feed into a microbial propagation system or bioreactor 190a. A growth media to facilitate the activation and/or growth of the genetically engineered microorganisms may be added as shown in FIG. 1. Alternatively, as shown in FIG. 2 all or a portion of the second process effluent with the high BOD concentration is fed into the propagation vessel 190a. If all the high BOD process effluent is added to propagation vessel 190a, and depending upon the size of the propagation vessel, the bioreactor 201 may not be needed. As stated previously, the genetically engineered microorganisms may also be activated prior to the contact with the high BOD process effluent. In this alternative embodiment propagation vessel 190a wherein the high BOD process effluent is added may not be needed. The hexanoic acid, process effluent with a high BOD concentration and genetically engineered microorganism that produces a cannabinoid are allowed to react until the desired BOD concentration is obtained. The cannabinoids and microbial biomass are removed from the reaction mass.

FIG. 2 shows two alternative methods for removing the cannabinoid from the reaction mass. The first option for removing the cannabinoid is use of a supercritical CO₂ extraction method 260, preferably a countercurrent multistage supercritical CO₂ extraction process similar to that described in FIG. 1. After the cannabinoids has been removed with the supercritical CO₂ extraction, the remaining reaction mass is separated into the process effluent with reduced BOD concentration and microbial biomass by any means known in the industry, preferably centrifugation 272. The second option for removing cannabinoids from the reaction mass of bioreactor 201 comprises contacting the reaction mass of bioreactor 201 with a material that retains a cannabinoid or lowers the moisture content to level which supercritical CO₂ extraction is viable such as activated charcoal, an ion exchange resin, diatomaceous earth, inert glass, or other materials and allowing the cannabinoid to react in reactor 271 with the retention/moisture content material and subjecting the reaction mass to a supercritical CO₂ extraction that removes the cannabinoid from the reaction mass 265. After the supercritical CO₂ extraction, the remaining reaction mass is separated in separator 273 into the process effluent with reduced BOD concentration, retention material and microbial biomass by any means known in the industry. The retention material may be reused.

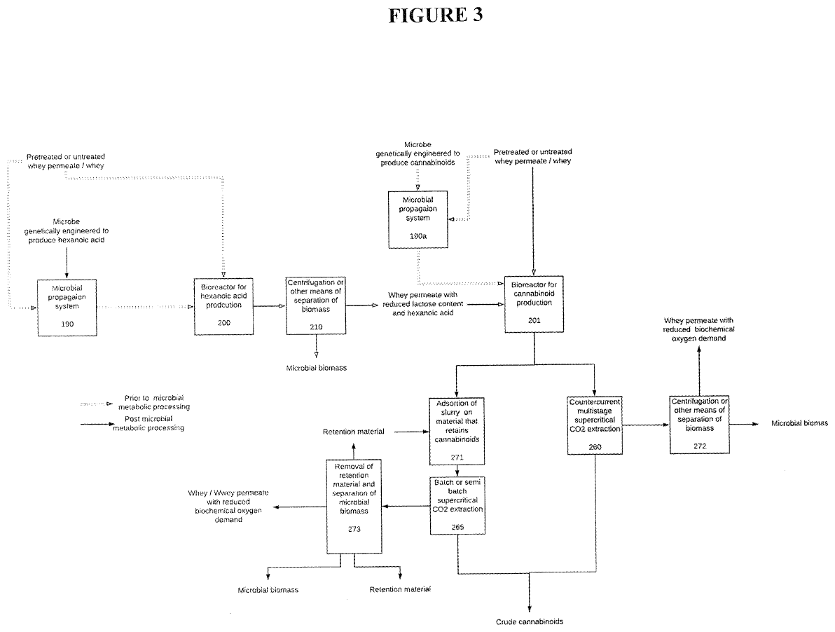

FIG. 3 shows the one embodiment of the present invention wherein a first process effluent with a high BOD concentration is treated with genetically engineered microorganisms to create a reaction mass comprising hexanoic acid, a process effluent with reduced BOD concentration and a microbial biomass. The microbial biomass is separated from reaction mass and the resulting reaction mass comprising the hexanoic acid and process effluent with reduced BOD concentration is added to a second process effluent with a high BOD concentration More specifically as shown in FIG. 3, a first process effluent with a high BOD concentration, such as whey or whey permeate, is fed into a reaction vessel, bioreactor 200, along with activated genetically engineered microorganisms that have been engineered to produce hexanoic acid. An example of a suitable genetically engineered microorganism that produces hexanoic acid is described in Cheon Y, et al., “A Biosynthetic Pathway for Hexanoic Acid Production in Kluyveromyces Marxianus,” J Biotechnol, July 2014 63(3): 223-24 (http://www.ncbi.nlm.nih.gov/pubmed/24768798) which is incorporated herein by reference. FIG. 3 further shows one aspect wherein the genetically engineered microorganisms that produce hexanoic acid are feed into a microbial propagation system or bioreactor 190. A growth media to facilitate the activation and/or growth of the genetically engineered microorganisms may be added as shown in FIG. 1 Alternatively, as shown in FIG. 3 all or a portion of the process effluent with the high BOD concentration is fed into the propagation vessel 190. If all the high BOD process effluent is added to propagation vessel 190 and depending upon the size of the propagation vessel, the bioreactor 200 may not be needed. As stated previously, the genetically engineered microorganisms may also be activated prior to the contact with the high BOD process effluent. In this alternative embodiment propagation vessel 190 wherein the high BOD process effluent is added may not be needed. The process effluent with a high BOD concentration and genetically engineered microorganisms that produces hexanoic acid are allowed to react until the desired BOD concentration is obtained. The microbial biomass is separated from the reaction mass by any means known in the art such as centrifugation 210. The reaction mass without the microbial biomass and comprising the hexanoic acid and the process effluent with reduced BOD concentration is then added to a reaction vessel or bioreactor 201.

As shown in FIG. 3, a second process effluent with a high BOD concentration, such as whey or whey permeate, is fed into a reaction vessel, bioreactor 201 along with activated genetically engineered microorganisms that have been engineered to produce a cannabinoid. An example of a suitable genetically engineered microorganism that produces a cannabinoid is described in U.S. Pat. No. 9,822,384 which is incorporated herein by reference. FIG. 3 further shows one aspect of this embodiment wherein the genetically engineered microorganisms that produce a cannabinoid are feed into a microbial propagation system or bioreactor 190a. A growth media to facilitate the activation and/or growth of the genetically engineered microorganisms may be added as shown in FIG. 1. Alternatively, as shown in FIG. 3 all or a portion of the second process effluent with the high BOD concentration is fed into the propagation vessel 190a. If all the high BOD process effluent is added to propagation vessel 190a and depending upon the size of the propagation vessel, the bioreactor 201 may not be needed. As stated previously, the genetically engineered microorganisms may also be activated prior to the contact with the high BOD process effluent. In this alternative embodiment propagation vessel 190a wherein the high BOD process effluent is added may not be needed. The hexanoic acid, process effluent with a high BOD concentration and genetically engineered microorganism that produces a cannabinoid are allowed to react until the desired BOD concentration is obtained. The cannabinoid and microbial biomass are removed from the reaction mass.

FIG. 3 shows two alternative methods for removing the cannabinoid from the reaction mass. The two options are similar to the two options previously described with reference to FIG. 2.

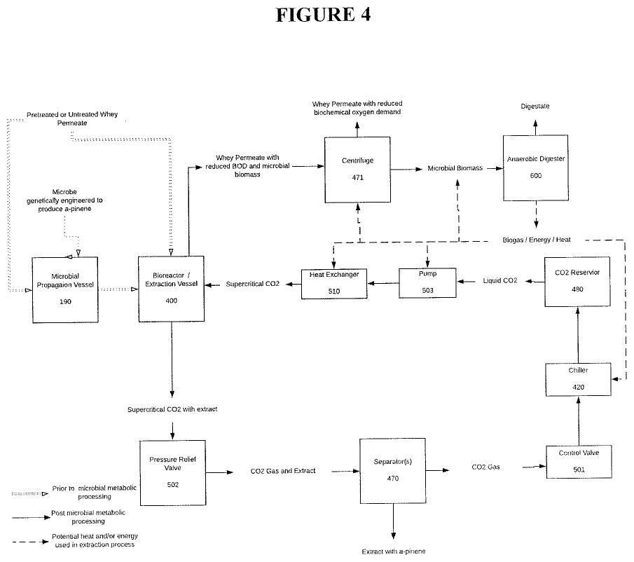

FIG. 4 shows one embodiment of the present invention wherein the microbial biomass is subjected to an anaerobic digestion process to produce a biogas such as CH₄ that is then used heat or power various aspect of the system. More specifically as shown in FIG. 4, a process effluent with a high BOD concentration, such as whey or whey permeate, is fed into a reaction vessel, bioreactor 400 along with activated genetically engineered microorganisms that have been engineered to produce a secondary metabolite, and specifically α-pinene. An example of a suitable genetically engineered microorganism that produces α-pinene is described in Yang J, et al, “Metabolic Engineering of Escherichia Coli for the Biosynthesis of Alpha-Pinene,” Biotechnol Biofuels, April 2013; 6(1):60 (http://biotechnologyforbiofuels.biomedcentral.com/articles/10.1186/1754-6834-6-60) which is incorporated herein by reference. FIG. 4 further shows one aspect wherein the genetically engineered microorganisms that produce α-pinene are feed into a microbial propagation system or bioreactor 190. A growth media to facilitate the activation and/or growth of the genetically engineered microorganisms may be added as shown in FIG. 1 Alternatively, as shown in FIG. 4 all or a portion of the process effluent with the high BOD concentration is fed into the propagation vessel 190. If all the high BOD process effluent is added to propagation vessel 190 and depending upon the size of the propagation vessel, the bioreactor 400 may not be needed. As stated previously, the genetically engineered microorganisms may also be activated prior to the contact with the high BOD process effluent. In this alternative embodiment propagation vessel 190 wherein the high BOD process effluent is added may not be needed. The process effluent with a high BOD concentration and genetically engineered microorganisms that produces α-pinene are allowed to react until the desired BOD concentration is obtained. The α-pinene from the reaction mass using a supercritical CO₂ extraction and separator 470. The separation of the α-pinene may occur by other any means commonly used in the industry. As shown in FIG. 4, the remaining reaction mass in bioreactor 400 is separated into the process effluent with reduced BOD concentration and microbial biomass by any means known in the industry, preferably centrifugation 471. The separated microbial biomass is subjected to anaerobic digestion 600 to produce a biogas such as CH₄ that is used to produce energy, i.e., heat and/or electricity to help run the processes and systems of the present invention.

The anaerobic digestion can be added to the processes and systems described in FIGS. 1-3.

The processes and systems of the present invention may be further modified. For example, the process effluent with the high BOD concentration may be pretreated prior to incorporation in the bioreactors with the genetically engineered microorganisms. Such pretreatment may comprise adjusting the pH to an acceptable or optimal level for microbial growth. Similarly, the temperature of the process effluent with the high BOD concentration may be adjusted to a temperature for acceptable or optimal level for microbial growth. In certain embodiments, when the process effluent comprises a large amount of lactose such as whey or whey permeate, the process effluent may be pretreated with microorganisms or enzymes such as β-galactosidases that cleave lactose into glucose and galactose prior to the addition of the genetically engineered microorganisms that produce the desired or target secondary metabolites selected from the group consisting of terpenoids, cannabinoids, cannabinoid producing enzymes, alkaloids or combinations thereof. This pretreatment to cleave lactose will allow genetically engineered microorganisms such as Saccharomyces cerevisiae to be used to produce secondary metabolites from high lactose containing process effluents such as whey and whey permeate.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A process for reducing biochemical oxygen demand (BOD) of a food or beverage processing effluent with a BOD greater than 750 mg/L comprising:
(i) contacting the process effluent with one or more microorganisms that have been genetically engineered to produce secondary metabolites wherein the secondary metabolites are selected from the group consisting of terpenoids, cannabinoids, cannabinoid producing enzymes, alkaloids or combinations thereof;
(ii) allowing the process effluent and microorganisms to react or ferment creating a reaction mass comprising the process effluent with a BOD less than 500 mg/L, the microorganisms and the secondary metabolites; and
(iii) removing the secondary metabolites from the reaction mass.

2. The process according to claim 1 wherein the process effluent is whey or whey permeate.

3. The process according to claim 1 wherein the microorganisms have been genetically engineered to produce a cannabinoid.

4. The process according to claim 1 wherein the secondary metabolites are removed with a supercritical CO₂ extraction using CO₂ obtained from step (ii).

5. The process according to claim 3 wherein hexanoic acid is added to step (ii).

6. The process according to claim 5 wherein the hexanoic acid is obtained from a process wherein a first a food or beverage processing effluent with a BOD greater than 750 mg/L is contacted with a genetically engineered microorganism that has been engineered to produce hexanoic acid.

7. The process according to claim 1 further comprising the step of separating the process effluent with a BOD less than 500 mg/L from the reaction mass.

8. The process according to claim 1 further comprising the step of separating the microorganisms from the reaction mass to create a biomass.

9. The process according to claim 8 comprising the steps of treating the biomass to anaerobic digestion to create a biogas and capturing the biogas to supply energy to the process equipment.
